(19) United States
(12) Patent Application Publication
(10) Pub. No.: US 2012/0107847 A1
(43) Pub. Date: May 3, 2012

(54) TESTING EFFICACY FOR CELIAC DISEASE

(75) Inventors: Maaike Johanna Bruins, Den Haag (NL); Luppo Edens, Rotterdam (NL)

(73) Assignee: DSM IP ASSETS B.V., Heerlen (NL)

(21) Appl. No.: 13/381,865

(22) PCT Filed: Jun. 24, 2010
(86) PCT No.: PCT/EP2010/058997

§ 371 Date: Dec. 30, 2011

(30) Foreign Application Priority Data
Jul. 2, 2009 (EP) 09008698.4
Sep. 28, 2009 (EP) 09171468.3
Feb. 23, 2010 (EP) 10154356.9

(51) Int. Cl. G01N 33/566; G06Q 90/00; G06Q 50/22; C12N 9/50

(52) U.S. Cl. 435/7.92; 435/219; 705/500

(57) ABSTRACT

We describe a method to determine effectiveness of a compound or composition in treatment of celiac disease or gluten intolerance, the method comprising:
administration of the compound or composition to one or more human patients who were on a gluten-free diet for 10 years or less, preferably 6 years or less;
challenge with gluten of the one or more human patients who were administered the compound or composition; and
determining whether there is a change in condition of celiac disease or gluten intolerance of the one or more patients that results from administration of the compound or composition and challenge with gluten; whereby no significant change in condition indicates that the compound or composition would be effective in treating celiac disease or gluten intolerance.

### FIELD OF THE INVENTION

The present invention relates to a method to determine the effectiveness of a compound or composition in treatment of celiac disease or gluten intolerance.

### BACKGROUND OF THE INVENTION

Celiac disease is a widely prevalent autoimmune disease of the small intestine which is caused by the ingestion of gluten. Gluten is a complex mixture of water insoluble, glutamine- and proline-rich proteins. Due to its very limited solubility in water and its extraordinary high levels of proline residues, the proteolytic degradation of gluten in the lumen of the human gastrointestinal tract is slow and specific protease-resistant peptides build up. Some of these protease-resistant peptides are thought to be toxic for patients suffering from celiac disease (Arentz-Hansen et al., J. Exp. Med. 6:337-342, 2000; Vader et al., Gastroenterol. 122:1729-1737, 2002). Ultimately, the toxicity results in an inflammation reaction leading to the destruction of the intestinal resorptive villi.

Many patients with untreated celiac disease develop antibodies to tissue transglutaminase. The enzyme tissue transglutaminase is released from among others endothelial cells upon its activation by, for example, inflammation reactions or wound formation. By cross-linking proteins (i.e., covalently linking glutamine with lysine residues in protein chains), tissue transglutaminase can repair wounds. However, the same enzyme also exhibits a deaminating activity by which glutamine residues, ubiquitously present in the toxic gluten peptides, are converted into glutamate residues. The latter conversion introduces extra negative charges on the peptide thereby further potentiating the T-cell stimulatory effect of toxic gluten epitopes.

Thus far, only a life-long gluten-free diet can effectively prevent the clinical symptoms in celiac disease patients. Unfortunately for celiac patients, gluten is a cheap protein with interesting application possibilities so that it is applied in a wide variety of foodstuffs including commercial soups, soy sauces, sauces, ice creams, potato chips and hot dogs. These broad application possibilities illustrate that complying with a gluten-free diet is difficult and that avoiding all gluten contaminations is nearly impossible. Gluten intolerant patients thus need detailed lists of foodstuffs to prevent the intake of the problematic gluten molecules because ingestion of gluten quantities as low as 50 mg per day may induce return of the clinical symptoms. It is therefore understandable that many efforts have been made to develop therapies that could simplify dietary compliance hereby reducing social pressure and improving the quality of life of celiac patients.

Currently various possible solutions are being investigated. In one promising approach an oral enzyme capable of hydrolyzing the toxic gluten epitopes is used. In this so-called enzyme-therapy, the patient consumes a small portion of a specific protease together with its meal. This oral protease hydrolyses, either in the stomach or further downstream, the gluten derived, toxic peptides into much smaller peptide fragments. Such much smaller fragments are no longer recognized as T-cell antigen and can be metabolized without problems. This approach is described in a number of patent applications such as WO 2002/45523, WO 2003/068170, WO 2005/027953 and WO 2008/115411. Apart from the microbial enzyme preparations specified in these patent applications, also duodenal enzyme extracts have been used (Cornell et al., Scand. J. Gastroenterol. 40:1304-1312, 2005).

Another approach that could lead to a possible therapy aims at reducing uncontrolled leakage of intestinal compounds. One of the physiological factors modulating intestinal permeability is zonulin. Zonulin is a protein involved in the opening of the intestinal epithelial tight junctions and its production is known to be upregulated in celiac patients. Therefore, inhibiting zonulin receptors could limit a paracellular leakage of toxic gluten fragments over the intestinal epithelium. Currently, the zonulin receptor inhibitor AT-100, is being developed as a potential candidate for the treatment of celiac disease by Alba Therapeutics Corporation (Paterson et al., Aliment. Pharmacol. Ther. 26:757-766, 2007).

In an approach that is being followed by ChemoCentryx, T cells of celiac patients homing to the digestive tract are addressed. It is known that trafficking of T cells to the small intestinal mucosa is controlled by a specific chemokine receptor. An antagonist should inhibit this T-cell homing process.

In yet another approach, tissue transglutaminase activity, known to potentiate the toxic effect of gluten epitopes by converting glutamine into glutamate residues, is inhibited. A number of institutes are working on this approach (e.g., US 2005/0244823 and Choi et al., Chem. Biol. 12:469-475, 2005). Other conceivable approaches, presumably more remote from clinical application, are listed in recent reviews by Lerner (Autoimmun. Rev. 9:144-147, 2010) and by Sollid and Lundin (Mucosal Immunol. 2:3-7, 2009).

Among celiac patients a high prevalence of various autoimmune disorders, especially type I diabetes, dermatitis herpetiformis, autoimmune thyroiditis, collagen diseases, autoimmune alopecia and autoimmune hepatitis, has been observed. Moreover, autoimmune diseases, in general, seem to result in increased intestinal permeability. The fact that celiac disease is occasionally accompanied by psychiatric and neurological symptoms, illustrates the far-reaching consequences that a combination of increased intestinal permeability with improper metabolism of proline-rich peptides may have. Therefore, there is an obvious need for therapies that can minimize the possibility of toxic gluten peptides entering the metabolic system of celiac patients or, in a broader sense, of individuals suffering from autoimmune disorders.

Despite a solid scientific basis in the form of many in vitro data, so far none of the above-described possible therapies seems to have yielded convincing clinical results. A major problem is that statistical analysis of the results obtained is often complicated by the poor response of celiac patients to gluten consumption. Therefore, until this very moment, it is not clear if the molecular concepts as proposed lack efficacy or that the tests used to diagnose remission of celiac disease are inappropriate. This is a particularly unsatisfying situation as it severely complicates further developments.

Obviously, a method by which the effectiveness of a compound or composition, and its mode of administration, towards treating gluten intolerance or a related disease can be unambiguously established would be of great value.

### SUMMARY OF THE INVENTION

The present invention relates to a method to determine effectiveness of a compound or composition and use of this compound or composition in treatment of celiac disease or gluten intolerance, the method comprising:
(a) administering the compound or composition to one or more human patients who were on a gluten-free diet for 10 years or less,
(b) challenging with gluten the one or more patients who were administered the compound or composition, and
(c) determining whether there is a change in condition of celiac disease or gluten intolerance of the one or more patients that results from administering the compound or composition and then challenging with gluten;
   whereby no significant change in condition indicates that the compound or composition would be effective in treating celiac disease or gluten intolerance.

The present invention also relates to a method to determine effectiveness of a compound or composition and the use of this compound or composition in treatment of celiac disease or gluten intolerance, the method comprising:
(a) selecting one or more human patients having celiac disease or gluten intolerance, wherein said patients
   (i) have a stable condition with respect to celiac disease or gluten intolerance and
   (ii) were on a gluten-free diet for 10 years or less;
(b) determining condition of illness of the one or more selected patients before administration of the compound or composition, and then challenge with gluten;
(c) administering the compound or composition to the one or more selected patients;
(d) challenging with gluten the one or more selected patients who were administered the compound or composition;
(e) determining condition of illness of the one or more selected patients after administration of the compound or composition, and then challenge with gluten; and
(f) comparing the condition of illness of the one or more selected patients before and after administration of the compound or composition, and then challenge with gluten;
   wherein a substantially unchanged condition of illness of the one or more selected patients after administration of the compound or composition and then challenge with gluten as compared to the condition of illness before administration of the compound or composition and then challenge with gluten indicates the compound or composition would be effective in treating celiac disease or gluten intolerance.

The present invention further relates to a method to improve analysis of data from a clinical trial; whereby one or more human patients are (a) administered a compound or composition, (b) challenged with gluten, and (c) diagnosed for a change in condition of celiac disease or gluten intolerance that results from administering the compound or composition and then challenging with gluten; the improvement being characterized by at least:
including in the clinical trial only the one or more patients who were on a gluten-free diet for 10 years or less, or
excluding from the clinical trial all of the one or more patients who were on a gluten-free diet for more than 10 years, or
including only data in the analysis obtained by diagnosing the one or more patients who were on a gluten-free diet for 10 years or less, or
excluding all data from the analysis obtained by diagnosing the one or more patients who were on a gluten-free diet for more than 10 years.

Determination or diagnosis of a condition of illness (e.g., celiac disease or gluten intolerance) after administration of the compound or composition and a challenge with gluten may be done after the trial is completed. Intermediate determination or diagnosis of a condition of illness can be done to stop the trial in case a patient strongly responses to the gluten challenge.

In the method according to the invention to determine effectiveness, a condition of illness after administration of the compound or composition and a challenge with gluten may be compared with a condition of illness after administration of a reference compound or composition, preferably a placebo and a challenge with gluten or only a challenge with gluten. According to another preferred embodiment in the method according to the invention to determine the effectiveness, a condition of illness before and after administration of the compound or composition and a gluten challenge may be compared with a condition of illness before or after administration of a placebo and a challenge with gluten or only a challenge with gluten.

Thus, advantageously, a change in condition of illness of one or more patients before and after administration of the compound or composition and a challenge with gluten is compared with a change in condition of illness of one or more patients before and after administration of a reference compound or composition, preferably a placebo, and challenge with gluten.

Preferably, one or more patients tested were on a gluten-free diet for more than 3 months, more preferably more than 6 months, and even more preferably at least 1 year. Preferably, the one or more patients tested were on a gluten-free diet for 8 years or less, and more preferably 6 years or less.

Suitable compounds or compositions to be used according to the invention comprise enzymes, preferably an enzyme which acts on gluten, more preferably a prolyl-specific endoprotease, a prolyl-specific (oligo)peptidase or a glutamine specific protease, most preferably a prolyl-specific endoprotease, or other compounds which can improve the intestinal barrier function preferably a zonulin receptor inhibitor or compound or composition having chemokine receptor antagonist action.

According to the present invention, a condition of illness can be determined by using known criteria for diagnosing celiac disease such as intestinal histopathology by Marsh classification, quantification of circulating antibodies such as tTGA antibody, measuring intestinal permeability using sugar test, presence and activity of T cells in blood and intestinal tissue, and symptoms analysis.

The effectiveness and use of the compound or composition are preferably done in a clinical trial with respect to celiac patients. This trial can be a treatment trial, quality of life trial, or compassionate use trial. Condition of illness, or change there between, may be determined or diagnosed in at least 5 human patients, preferably at least 10 human patients, and more preferably at least 20 human patients and most preferably at least 50 human patients.

The present invention also allows a novel interpretation of existing data on clinical trials. By using the results from (already public) data on the effectiveness of tested compounds and taking only the results of the patients who use a gluten-free diet for 10 years or less, preferably 8 years or less, more preferably 6 years or less, the effectiveness of a compound or composition in treatment of celiac disease or gluten intolerance can be determined or re-evaluated. So the present invention provides an improved interpretation of data of clinical trials designed for testing the effectiveness of a compound or composition in treatment or prevention of celiac disease or gluten intolerance, by focusing on the results obtained with one or more patients who were on a gluten-free diet for 10 years or less, preferably 8 years or less, and more preferably 6 years or less.

The present invention also relates to a health claim, disease reduction claim, or label for a compound or composition for treatment of celiac disease or gluten intolerance which is based on the interpretation of a clinical trial for testing the effectiveness of a compound or composition in treatment of celiac disease or gluten intolerance, by using the results from one or more human patients who were on a gluten-free diet for 10 years or less, preferably 8 years or less, and more preferably 6 years or less, preferably by using only the results from one or more patients who were on a gluten-free diet for 10 years or less, preferably 8 years or less, and more preferably 6 years or less. This health claim, disease reduction claim, or label can be used to advertise or promote a compound or composition for treatment or celiac disease or gluten intolerance.

The present invention also discloses exclusion or inclusion criteria in a clinical trial for testing a compound or composition for treatment of celiac disease or gluten intolerance which comprise patients who use a gluten-free diet for 10 years or less, preferably 8 years or less, more preferably 6 years or less.

### DETAILED DESCRIPTION OF SPECIFIC EMBODIMENTS

The only proven effective treatment of celiac disease is a life-long gluten-free diet. So far no medication is available for preventing or undoing the toxic effects of gluten in celiac patients. Although considerable experimental data are available from clinical trials on celiac patients, the interpretation of these data does not allow statistically significant correlations between the treatment of the disease and the compound or composition used. Thus, a simple and straightforward correlation between the symptoms observed and the administration of an active compound could not be made. However, after a careful analysis of the results of the present clinical tests, the present inventors have reached the surprising conclusion that only recently diagnosed celiac patients can provide the biochemical and histopathological responses that are required for a proper assessment of the data obtained. By recently diagnosed celiac patients are meant patients that are ten years or less, preferably eight years or less, more preferably six years or less on a gluten-free diet. By including patients that have been on a gluten-free diet for longer times, the outcomes of clinical tests become blurred and they make it difficult to draw conclusions. Thus, the present inventors pose the hypothesis that for obtaining maximal discriminative power, only those patients that can provide the desired response should be incorporated in the trial. These patients are less than ten, preferably less than eight years, more preferably less than six years on a gluten-free diet. This six-, eight- or ten-year period mentioned in relation to the period for a gluten-free diet may vary from patient to patient but it is believed to be correct in general. It is believed that, upon gluten consumption, patients that have been on a gluten-free diet for many years, for example more than 10 years, will show no or hardly any of the characteristic symptoms after a gluten challenge. In the present context gluten consumption or gluten challenge can range from 50 milligrams to tens of grams a day for a short period which implies daily gluten consumption for at least one week.

Although the above theory is believed to be true, the present invention does not rely on the correctness of this theory as it is only given to provide a possible explanation for the results found according to the invention. Although, in hindsight, this theory might seem simple, a lot of biochemical and histopathological testing and clinical trials have been, and are being done, without mentioning, explaining, or suggesting the present criteria or findings. The latter statement is easily proven by the eligibility criteria specified for candidates for Phase 2 celiac studies [cf. http://clinicaltrials.gov] Although minimal periods for a strict adherence to a gluten-free diet are specified, neither Alvine Pharmaceutical, nor Alba Therapeutics Corporation nor Chemocentryx specified maximum periods for gluten-free diets in their relevant enrolment procedures.

Up to now it is assumed that there is no correlation between relapse time after gluten challenge and time spent on a gluten free diet (Kumar et al., Gut, 1979, 20, pp. 743-749) let alone the extent of response on the gluten challenge and time spent on a gluten free diet.

According to one embodiment of the present invention, only the data of patients on gluten-free diets for ten years or less, preferably eight years or less, more preferably six years or less, are included in analysis of a clinical trial aimed at testing effectiveness of a therapy for celiac patients. According to another embodiment, in a clinical trial aimed at testing therapies for celiac patients, the data of patients not satisfying such requirements are not included in analysis of a clinical trial aimed at testing effectiveness of a therapy for celiac patients. According to yet another embodiment, the inclusion and/or exclusion criteria for recruiting participants in a clinical trial aimed at testing effectiveness of a therapy for celiac patients specify such requirements.

By “gluten-free diet” is meant essentially free of ingredients derived from gluten-containing cereals: wheat (including kamut and spelt), barley, rye, and triticale as well as use of gluten as a food additive in the form of a flavouring, stabilizing, or thickening agent. Additionally, the diet may exclude oats. Since it would be practically impossible to maintain a diet containing no or zero gluten because of trace amounts or contaminants introduced during food processing, a gluten-free diet permits foods containing up to 100 ppm of gluten, preferably up to 20 ppm of gluten, to keep gluten ingestion below a 50 mg/day threshold level.

Visakorpi (“Definition of coeliac disease in childhood”, Coeliac Disease: Proceedings of the Second International Coeliac Symposium, pp. 10-16, 1974) and Rolles et al., Archives of Disease in Childhood, 50, pp 259-263, 1975) suggest gluten challenge for children, however according to the present invention preferably patients at least 16 years old, more preferably at least 18 years old, or even more preferably at least 21 years old are tested for effectiveness of a therapy for celiac disease or gluten intolerance. Although patients less than 16 years of age may meet the criterion of being on a gluten-free diet for 10 years or less, 8 years or less, or 6 years or less, celiac disease or gluten intolerance may disappear during adolescence. Moreover, gluten challenge should not be used in youngsters if not absolutely necessary because such tests might result in additional negative effects. Although patients aged at least 16, 18 or 21 years may be tested for effectiveness of a therapy for celiac disease or gluten intolerance, the compounds or compositions that are found to be effective can later be used for patients of all ages including youngsters.

By “challenge with gluten” is meant administering at least 50 mg, preferably at least 1 g, more preferably at least 5 g, and even more preferably at least 10 g of gluten per day for at least 7 days, preferably at least 14 days, more preferably at least 30 days, and even more preferably at least 90 days.

In the United States, the Food and Drug Administration (FDA) requires numerous clinical trials to prove a drug's safety and efficacy in treating a given disease or condition. If satisfied that the drug is safe and effective, the drug's manufacturer and the FDA agree on specific language describing dosage, route and other information to be included on the drug's label. More detail is included in the drug's package insert. Both prescription and over-the-counter medicines must be approved before they can be sold in the U.S.

Health claims or disease (risk) reduction claims on (functional) food labels are claims by manufacturers of food products that their food will reduce the risk of developing a disease or condition which are based on scientific research. Health claims or disease (risk) reduction claims are messages printed on the food label to describe a relationship between a food substance or nutrient and a health-related condition.

The present invention also relates to a health claim, disease reduction claim, or label for a compound or composition for treatment of celiac disease or gluten intolerance or labeling a compound or composition for treatment of celiac disease or gluten intolerance which is based on interpretation of the data generated in a clinical trial designed for testing effectiveness of a compound or composition in treatment of celiac disease or gluten intolerance, if only results from one or more patients on a gluten-free diet for 10 years or less, preferably 8 years or less, and more preferably 6 years or less are used. Such health claim, disease reduction claim, or label can be used to advertise or promote a compound or composition for treatment of celiac disease or gluten intolerance.

By label is meant a display of written, printed, or graphic matter (or the electronic version thereof) upon the immediate container of the compound or composition for the treatment of celiac disease or gluten intolerance, including the outside container and wrapper or accompanying the compound or composition for the treatment of celiac disease or gluten intolerance.

“Labeling” is a method of advertisement or promotion. This includes the “label” and other written, printed, or graphic matter (or the electronic version thereof) upon a compound or composition for the treatment of celiac disease or any of its containers or wrappers, or accompanying the compound or composition for the treatment of celiac disease or gluten intolerance. Examples of “labeling” include brochures, booklets, mailing pieces, detailing pieces, file cards, bulletins, including the electronic version thereof, etc.

Therapeutic or disease reduction claims specifically approved by the FDA are included in the drug (compound or composition for treating celiac disease) labeling. The approved labeling is the official source from which all useable information for advertising and promotion is derived.

In the present text the word “effectiveness” is interchangeably used for “efficacy” in the meaning of the capability for generating beneficial therapeutic effect.

Diagnosis of celiac disease can be assisted by several tests such as blood tests, endoscopy, pathology or other tests [see: http://en.wikipedia.org/wiki/Coeliac_disease].

Stable condition or stable condition of illness means that patients

have confirmed diagnosis of celiac disease (in the past)

are on a gluten-free diet for at least 3 months, preferably at least 6 months, or more preferably at least 1 year; and

do not have severe clinical signs of celiac disease, for example having a serum anti-tissue Transglutaminase antibody titer of less than 21 aU/mL, preferably of 6 aU/mL or less, or more preferably less than 3 aU/mL or a Marsh score of 3a or less, preferably of 2 or less, or more preferably are free of signs of celiac disease (e.g., having a Marsh score of 0 or 1).

Antibody Testing (Blood Test)

Serological blood tests are the first-line investigation required to make a diagnosis of celiac disease. Serology for anti-tTG antibodies has superseded older serological tests and has a high sensitivity (99%) and specificity (>90%) for identifying celiac disease. Modern anti-tTG assays rely on a human recombinant protein as an antigen (Sblattero et al., Am. J. Gastroenterol. 95:1253-1257, 2000)

HLA Genetic Typing (Blood Test)

Antibody testing and HLA testing have similar accuracies (Hadithi et al., Ann. Intern. Med. 147:294-302, 2007).

Endoscopy

An upper endoscopy with biopsy of the duodenum (beyond the duodenal bulb) or jejunum is performed. It is important for the physician to obtain multiple samples (four to eight) from the duodenum.

Pathology

Determination of the occurrence of characteristic gluten induced histopathological changes in small intestinal biopsies, including intraepithelial lymphocytosis, crypt hyperplasia and villous atrophy, according to the Modified Marsh criteria (Rostami et al., Am. J. Gastroenterol. 94:888-894, 1999).

Modified Marsh Criteria

Marsh 0: normal mucosal architecture
Marsh 1: normal mucosal architecture with an infiltration of villous epithelium by lymphocytes; more than 30 lymphocytes per 100 enterocytes
Marsh 2: intraepithelial lymphocytosis and crypthyperplasia
Marsh 3a: intraepithelial lymphocytosis, crypthyperplasia and partial villous atrophy (villus-crypt ratio was less than 1:1)
Marsh 3b: intraepithelial lymphocytosis, crypthyperplasia and subtotal villous atrophy
Marsh 3c: intraepithelial lymphocytosis, crypthyperplasia and total villous atrophy

In medical research, clinical trials are conducted to allow safety and efficacy data to be collected for new drugs or devices. These trials can only take place once satisfactory information has been gathered on the quality of the product and its non-clinical safety, and Health Authority/Ethics Committee approval is granted in the country where the trial is taking place [http://en.wikipedia.org/wiki/Clinical_trial].

Depending on the type of product and the stage of its development, investigators enroll healthy volunteers and/or patients into small pilot studies initially, followed by larger scale studies in patients that often compare the new product with the currently prescribed treatment. As positive safety and efficacy data are gathered, the number of patients is typically increased. Clinical trials can vary in size from a single center in one country to multicenter trials in multiple countries.

Many of the below terms are taken from Glossary of Clinical Trials Terms, [http://clinicaltrials.gov/ct2/info/glossary] from U.S. National Institutes of Health (NIH).NIH organizes trials into five (5) different types:

Prevention trials: look for better ways to prevent disease in people who have never had the disease or to prevent a disease from returning. These approaches may include medicines, vitamins, vaccines, minerals, or lifestyle changes.

Screening trials: test the best way to detect certain diseases or health conditions.

Diagnostic trials: conducted to find better tests or procedures for diagnosing a particular disease or condition.

Treatment trials: test experimental treatments, new combinations of drugs, or new approaches to surgery or radiation therapy.

Quality of life trials: explore ways to improve comfort and the quality of life for individuals with a chronic illness (a.k.a. Supportive Care trials).

Compassionate use trials: provide experimental therapeutics prior to final FDA approval to patients whose options with other remedies have been unsuccessful. Usually, case-by-case approval must be granted by the FDA for such exceptions.

A distinction is made between the preclinical and clinical trial phases.

Preclinical: Refers to the testing of experimental drugs in the test tube or in animals—the testing that occurs before a trial is performed in humans.

Clinical trial: A clinical trial is a research study to answer specific questions about vaccines or new therapies or new ways of using known treatments. Clinical trials (also called medical research and research studies) are used to determine whether new drugs or treatments are safe and effective. Carefully conducted clinical trials are the fastest and safest way to find treatments that work in people. Trials are in four phases:

Phase I trials: Initial studies to determine the metabolism and pharmacologic actions of drugs in humans, the side effects associated with increasing doses, and to gain early evidence of effectiveness; may include healthy participants and/or patients.

Phase II trials: Controlled clinical studies conducted to evaluate the effectiveness of the drug for a particular indication or indications in patients with the disease or condition under study and to determine the common short-term side effects and risks.

Phase III trials: Expanded controlled and uncontrolled trials after preliminary evidence suggesting effectiveness of the drug has been obtained, and are intended to gather additional information to evaluate the overall benefit-risk relationship of the drug and provide and adequate basis for physician labeling.

Phase IV trials: Post-marketing studies to delineate additional information including the drug's risks, benefits, and optimal use.

Inclusion/exclusion criteria: The medical or social standards determining whether a person may or may not be allowed to enter a clinical trial. These criteria are based on such factors as age, gender, the type and stage of a disease, previous treatment history, and other medical conditions. It is important to note that inclusion and exclusion criteria are not used to reject people personally, but rather to identify appropriate trial participants and to ensure their safety.

Exclusion criteria for method according to the invention could be:

Use of any immunoregulatory drug within the last 6 months; or

Use of any anticoagulant drug; or

Clinically suspected bleeding tendency; or

Pregnancy or breast feeding; or

Presence of any concurrent active infection; or

IgA deficiency; or

Refractory celiac disease or serious/severe complications of celiac disease; or

Chronic active gastrointestinal disease other than celiac disease; or

History of any medically significant condition considered by the enrolling physician to adversely affect participation in the trial.

Placebo: A placebo is an inactive pill, liquid, or powder that has no treatment value. In clinical trials, experimental treatments are often compared with placebos to assess the treatment's effectiveness.

Diagnosing celiac disease is difficult and may involve immunological criteria as well as histopathological analysis of duodenal biopsies, sometimes after a gluten challenge. Such gluten challenges are typically applied in individuals under investigation for suspected celiac disease. Over the past decades, many trials incorporating such gluten challenges have been published, not only with adults but also with children. In the latter experiments, typically at least 25 children consuming a few grams of gluten per day for periods of a few months up to one year were involved. The design of such tests carried out with adults was more heterogeneous and many different conditions were used—small and large groups, gluten dosages varying from a few grams to thirty grams of gluten per day in single boluses and during periods up to 14 months. The wide scatter in the methods used and the fact that such trials continue to be published illustrate the complexity of a proper diagnosis of celiac disease. Clear guidelines regarding duration of the challenge, the gluten dose used and the monitoring parameters are missing and still under debate. Even judging deterioration of the intestinal mucosa according to the Marsh criteria, a major requirement for establishing a proper diagnosis, is far from ideal as the observation is difficult to interpret and subject to individual judgment.

It is against this background that the efficacies of therapies conceived for treating celiac disease have to be interpreted. On top of that, to the best of their knowledge the inventors are aware of only a single clinical study of which hard numerical data have been published: an enzyme therapy using animal duodenal extracts (Cornell et al., Scand. J. Gastroenterol. 40:1304-1312, 2005). In this study, 21 celiac patients with biopsy—proven celiac disease were challenged with less than 1 gram of gluten per day for a period of two weeks in a randomized, placebo-controlled clinical trial. After a challenge of two weeks with either the extract or the placebo, the groups were crossed over for the extract or placebo interventions and symptoms were recorded for the next twelve weeks.

Despite the observation that those patients who showed moderate to severe symptoms to the challenge, seemed to benefit from the therapy, the following citation by the authors is illustrative for the situation: “In fact, the low level of symptoms in most of the participants during placebo or enzyme therapy limited our capacity to detect any protection by enzyme therapy during gluten challenge as measured by overall symptom scores in the entire cohort”.

Also Alvine Pharmaceuticals, Inc. is quite involved in investigating enzyme therapy for celiac. However, according to their press releases (www.alvinepharma.com) their ALV003 oral protease therapy has not progressed beyond Phase 1 trials in which the safety and tolerability of the product in healthy volunteers is confirmed.

In contrast with Alvine Pharmaceutical, Alba Therapeutics Corporation (www.albatherapeutics.com) is focussing on their zonulin receptor inhibitor AT-1001 (larazotide acetate). Having finished a Phase 1 safety assessment of the compound and a Phase IIa double-blind, placebo-controlled, dose ranging, safety study, they have entered into a Phase IIb, double-blind, placebo-controlled, dose ranging study to determine the efficacy of the compound in 184 adults with controlled celiac disease during a gluten challenge. According to a statement of the company's CEO early 2009, “The data are promising and will be publicly presented at upcoming scientific meeting this spring”. However, until this very day relevant detailed numerical information has not been published so far.

Chemocentryx (www.chemocentryx.com) is currently evaluating their Traficet-EN product in a Phase II clinical trial in patients with celiac disease. No experimental data from this study have been published.

### BRIEF DESCRIPTION OF THE DRAWINGS

FIG. 1: The relationship between years on gluten-free diet and the intestinal histopathological response (Marsh) to 2-weeks of 7 g daily gluten intake in patients receiving placebo (A) or AN-PEP treatment (B).

FIG. 2: The relationship between years on gluten-free diet and the serum tTGA (tissue transglutaminase IgA) response to 2-weeks of 7 g daily gluten intake in patients receiving placebo (A) or AN-PEP treatment (B).

FIG. 3: The relationship between years on gluten-free diet and the serum AGA (anti-gliadin IgA) response to 2-weeks of 7 g daily gluten intake in patients receiving placebo (A) or AN-PEP treatment (B).

FIG. 4: The relationship between years on gluten-free diet and the serum AGG (anti-gliadin IgG) response to 2-weeks of 7 g daily gluten intake in patients receiving placebo (A) or AN-PEP treatment (B).

FIG. 5: Treatment effect of AN-PEP on tTGA antibody response versus placebo when selecting patients on a gluten-free diet for more than 6 years (A) or selecting patients on a gluten-free diet for 6 years or less (B).

FIG. 6: Treatment effect of AN-PEP on DGP-tTG antibody response versus placebo when selecting patients on a gluten-free diet for more than 6 years (A) or when selecting patients on a gluten-free diet for 6 years or less (B).

FIG. 7: Design of the clinical trial.

FIG. 8: Rank correlations (r) between subjects' years on gluten-free diet and intestinal histopathological Marsh score, at 2 weeks of gluten intake in the placebo group. The correlation was significant (p=0.027).

FIG. 9: Rank correlations (r) between subjects' years on gluten-free diet and serum tTGA antibodies, at 2 weeks of gluten intake in the placebo group. The correlation was significant (p=0.018).

FIG. 10: Rank correlations (r) between subjects' years on gluten-free diet and serum AGA antibodies, at 2 weeks of gluten intake in the placebo group. The correlation was not significant (p=0.086).

FIG. 11: Rank correlations (r) between subjects' years on gluten-free diet and serum AGG antibodies, at 2 weeks of gluten intake in the placebo group. The correlation was significant (p<0.0001).

FIG. 12: Rank correlation (r) between subjects' years on gluten-free diet and serum DGP-tTG antibodies, at 2 weeks of gluten intake in the placebo group. The correlation was significant (p=0.04).

### MATERIALS AND METHODS

AN-PEP

In WO 2005/027953, a food-grade Aspergillus niger proline-specific endoprotease (also named AN-PEP) is described for in vivo treatment of gluten-containing food.

AN-PEP activity measurement

AN-PEP protease activity was tested using CBZ-Gly-Pro-pNA (Bachem, Bubendorf, Switserland) as a substrate at 37° C. in a citrate/disodium phosphate buffer pH 4.6. The reaction products were monitored spectrophotometrically at 405 nM. The increase in absorbance at 405 nm in time is a measure for enzyme activity. A Proline Protease Unit (PPU) is defined as the quantity of enzyme that releases 1 μmol of p-nitroanilide per minute under the conditions specified and at a substrate concentration of 0.37 mM Z-Gly-Pro-pNA.

AN-PEP activity in relation to the gluten consumed

AN-PEP dosage in the clinical trial was in accordance with the validated in vitro data described by Mitea et al. (Gut 57:25-32, 2008). Per gram of dietary protein present 20 PPU were dosed.

Composition and shelf stability of the AN-PEP containing toast topping and its placebo

The enzyme containing toast topping and its placebo product were carefully prepared to obtain products with similar taste, pH, color and texture. Because the enzyme concentrate was slightly brown, the color of the placebo (with distilled water replacing the enzyme concentrate) was corrected by adding a coloring agent. The enzyme concentrate turned out to be virtually tasteless and adding citric acid with sucrose and a saccharine solution, yielded quite acceptable taste profiles. In both products the final pH values were adjusted to 3.7. To guarantee that during the whole experiment similar enzyme activities were consumed and the microbiological safety was guaranteed, the enzyme product and its placebo were continuously monitored. Over a period of one year storage at 6 degrees C., no microbial contaminations were noted and the enzymatic activity remained stable around a value of 9 PPU/g. The precise compositions are specified in Table 1.

TABLE 1
Composition of AN-PEP and control topping.

| | AN-PEP topping | Placebo |
|---|---|---|
| | (wt %) | (wt %) |
| AN-PEP enzyme concentrate | 81.5 | |
| Distilled water | | 81.5 |
| Plantex MDA31 (coloring agent) | | 0.06 |
| Sucrose | 8.2 | 8.2 |
| Saccharine solution (400 mg/l | 8.2 | 8.2 |
| saccharine plus 4000 mg/l | | |
| cyclamate) | | |
| Citric Acid | 0.4 | 0.4 |
| Potassium sorbate | 0.08 | 0.08 |
| Sodium benzoate | 0.31 | 0.31 |
| Xanthane gum (Keltrol RD) | 1.23 | 1.23 |

### Example 1

### Enzyme Therapy and its Clinical Responses in Celiac Subjects on a Gluten Diet

Because of their unusually high content of proline, gluten molecules are poorly degraded by enzymes in the gastrointestinal tract. After ingestion, these partly degraded gluten proteins reach the small intestine. Such proline-rich protein fragments are particularly toxic to celiac patients as they will induce T-cell responses causing an inflammatory response in the small intestine.

Initial in vitro studies showed that the proline-specific endoprotease AN-PEP is highly effective in gluten molecules that are toxic to celiac patients by cleaving after the proline residues (Stepniak et al., Am. J. Physiol. Gastroenterol. Liver Physiol. 291:G621-G629, 2006). Moreover, the enzyme is stable and active under gastric conditions (Stepniak et al.). It was also shown that in vitro incubation of a digest of gluten with this AN-PEP enzyme was able to almost fully reduce immune T-cell responses to gluten (Stepniak et al.). Later studies showed that in a simulated gastrointestinal tract model the AN-PEP enzyme was able to digest the toxic gluten sites of a gluten-containing test meal in the stomach and particularly in the small intestine (Mitea et al., Gut 57:25-32, 1008).

The aim of the present study was to evaluate whether oral intake of AN-PEP can mitigate the toxic effects of gluten ingestion in patients with celiac disease. Histopathology of intestinal biopsies and immune markers in blood serum was used to establish the condition of illness prior to, during and after the test. Histopathology in intestinal biopsies was expressed according to the Marsh classification and immune markers in serum included a variety of celiac disease-specific antibodies.

Subjects

Nineteen celiac patients were recruited at the VU University Medical Center Amsterdam, the Netherlands. Inclusion criteria for study entry were: confirmed diagnosis of celiac disease in the past followed by a stable clinical picture (stable condition) on a strict gluten-free diet for at least 1 year. Patients should have a confirmed diagnosis of celiac disease in the past as defined by Marsh 3b/3c on biopsies (intraepithelial lymphocytosis, crypt hyperplasia and subtotal or total villous atrophy) as well as detectable celiac disease-specific serum IgA antibodies against endomysium or tissue transglutaminase (EMA, tTGA) while using a gluten-containing diet. Stable celiac disease was defined as clinically normalised while using a gluten-free diet, i.e., meaning normalised villous architecture (Marsh 0/1) and no detectable EMA and low tTGA (<4 U/mL) prior to the start of the study. Other inclusion criteria for study entry were: between 18-70 years of age; willing to undergo all protocol related assessments and visits (including up to 3 separate esophago-gastro-duodenoscopies with multiple biopsies taken each time from the descending duodenum); having read the information provided on the study and given written consent; and use of adequate contraception for female participants at fertile age. Reasons for exclusion were: use of any immunoregulatory drug within the last 6 months; use of any anticoagulant drug; clinically suspected bleeding tendency; pregnancy or breast feeding; presence of any concurrent active infection; and IgA deficiency. Subjects visited the study site one week before start of the study for an intestinal biopsy to confirm a normal villous architecture (Marsh 0/1). Subjects having a normal villous architecture were enrolled in the study. The study was approved by the Medical Ethics Committee of the VU University Medical Center and conducted in accordance with the Helsinki Declaration. A written informed consent was obtained from each subject before enrolment.

The study consisted of three phases. During the 1^{st} safety phase, all patients were asked to consume daily five “English toasts” (corresponding with ˜7 g gluten, Bolletje®, The Netherlands) with 18.5 g enzyme topping for breakfast for 2 weeks. The topping contained 177 Proline Protease Units (see Materials and Methods for unit definition) AN-PEP enzyme. During a 2^{nd} two-week wash-out period, the patients consumed their usual gluten-free meals. In the 3^{rd} randomization phase, after the wash-out period, patients were randomized in a double-blind fashion to the same daily 7 g gluten-containing breakfast with either 18.5 g AN-PEP topping (n=7) or 18.5 g placebo topping (n=7) for 2 weeks. The compositions of the toppings are given in Table 1 in the Materials and Methods section. Subjects were only allowed to consume a single glass of water with their breakfast.

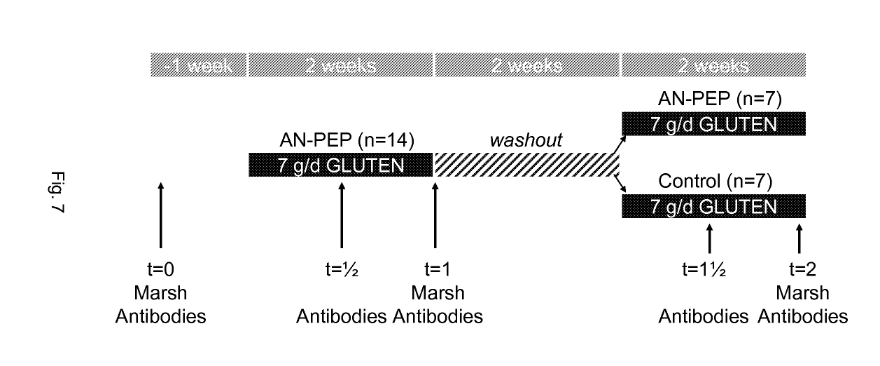

During the study, the patients visited the outpatient clinic of the medical center five times (see FIG. 7 for clinical trial design). Blood was sampled before (baseline, t=0), during (t=½) and after the safety phase (t=1) as well as during (t=1½) and after the randomization phase (t=2). Serum was used to determine celiac-specific antibodies as anti-endomysium IgA (EMA), tissue transglutaminase IgA (tTGA), and anti-gliadin IgA (AGA) and IgG (AGG) antibodies. Recently, a sensitive assay with high diagnostic accuracy for celiac disease has become available detecting IgA and IgG for both tissue transglutaminase (tTG) and deamidated gliadin peptides (DGP), i.e., DGP-tTG (Lewis NR, Scott BB. Aliment Pharmacol Ther 2009). All antibodies were determined by ELISA. Duodenal biopsies were taken before the start of the study (t=0), after the safety phase (t=1) as well as after the randomization phase (t=2). Twelve spike-biopsy samples were obtained through oesophago-gastro-duodenoscopy from the descending duodenum. Biopsies were submitted to histopathological examination and scored by a pathologist according to the modified UEGW (United European Gastroenterology Week) Marsh criteria 2001.

Results

Study Population

Nineteen subjects were recruited. Three subjects were not included in the study as their villous architecture was not normalised on a gluten-free diet (1× Marsh score of M2, 2× Marsh scores of M3A). The remaining 16 subjects entered the study. The median age of the subjects (4 males, 12 females) was 55 (20-68) years. The median age at which they were diagnosed with celiac disease was 44 (0-62) years. The median time on a gluten-free diet was 8 (2-40) years. The median time between symptoms and diagnosis of celiac disease was 20 (0-64) years. The HLA status was DQ2 heterozygous (n=11), DQ2 homozygous (n=2), DQ2/8+heterozygous (n=1), and unknown (n=2).

Safety Phase (2-Week Consumption of Gluten Plus AN-PEP Topping)

Sixteen subjects entered into the study. At the end of the safety phase, three of sixteen subjects showed elevated serum anti-gliadin IgA (AGA) and 1 subject showed a dubious increase in anti-gliadin IgG (AGG), see Table 2. Also, AGA antibodies significantly increased during the safety phase. However, anti-endomysium IgA (EMA), tissue transglutaminase IgA (tTGA), and IgG (AGG) did not significantly increase during the safety phase. The intestinal biopsies of patients at baseline had either Marsh 0 or Marsh I scores. Two subjects deteriorated 2 Marsh scales but did not show positive antibodies. The latter two subjects were excluded from the study. The change in Marsh in the total group during the safety phase was not significant.

Wash-Out Period

Fourteen subjects entered into the 3^{rd} phase. During this period, all patients returned for a period of two weeks to their original gluten-free meals and no toasts or AN-PEP was consumed.

Randomization Phase (Gluten Challenge Plus Either AN-PEP or Placebo Topping)

Quite surprisingly during the randomization phase (from baseline to end of the phase), only few patients deteriorated during the 2-weeks of gluten challenge. The intestinal histopathology (Marsh score) did not significantly change in the total group, neither in the control group, nor in the AN-PEP group. The mean increase in Marsh score during the randomization period in the total group was only 0.4±0.8 on a scale of 4 as compared to baseline. Also, the increases in serum antibodies (tTGA, AGA, AGG, and DGP-tTG) were not significant during the randomization phase while EMA remained negative in all subjects. In the total group, the mean (±SD) increase in tTGA, AGA, AGG was 0.4±0.9, 4.0±11.9, 5.0±11.0, and 5.2±11.2 aU/mL, respectively. No treatment effects of AN-PEP were observed in the randomization phase, neither for intestinal histopathology, nor for serum antibodies. The data obtained are shown in Table 2.

TABLE 2
Responses of the 16 subjects included in the study responded to gluten by
clinical signs (intestinal histopathology as measured by Marsh score and the celiac-
disease specific serum antibodies EMA, tTGA, AGA, AGG, and DGP-tTG).

| | Before | Safety | | | | Randomization | End of randomization | |
|---|---|---|---|---|---|---|---|---|
| | start | phase | End of safety phase | | | phase | phase | |
| | Marsh | Gluten + | Marsh | | | Gluten + | Marsh | |
| Pt | score | Treatment | score | Antibodies | Washout | Treatment | score | Antibodies |
| 1 | 1 | AN-PEP | 0 | | | Control | 1 | |
| 2 | 1 | AN-PEP | 2 | | | AN-PEP | 1 | |
| 3 | 0 | AN-PEP | 1 | | | AN-PEP | 1 | DGP-tTG + |
| 4 | 0 | AN-PEP | 3a | | Excluded | | | |
| 5 | 0 | AN-PEP | 1-2 | AGA: +¹ | | Control | 3a | AGA: ++ |
| | | | | | | | | AGG: + |
| 6 | 1 | AN-PEP | 1 | | | Control | 3a | |
| 7 | 1 | AN-PEP | 0 | | | Control | 1 | tTGA + |
| | | | | | | | | DGP-tTG |
| | | | | | | | | ++ |
| 8 | 0 | AN-PEP | 1 | AGA: +/− | | AN-PEP | 1 | AGA: +/− |
| | | | | | | | | AGG: + |
| | | | | | | | | DGP-tTG + |
| 9 | 1 | AN-PEP | 2 | AGA: + | | AN-PEP | 3a | AGA: +/− |
| 10 | 0 | AN-PEP | 0 | | | Control | 0 | |
| 11 | 0 | AN-PEP | 2 | AGG: +/− | Excluded | | | |
| 12 | 1 | AN-PEP | 1 | | | AN-PEP | 1 | |
| 13 | 0 | AN-PEP | 0 | | | Control | 0 | |
| 14 | 0 | AN-PEP | 0 | | | AN-PEP | 0 | |
| 15 | 0 | AN-PEP | 0 | | | AN-PEP | 0 | |
| 16 | 0 | AN-PEP | 0 | | | Control | 1 | |

¹An antibody value of +/−, +, ++ and +++ is defined as follows:

| | | | | |
|---|---|---|---|---|
| | tTGA (aU/mL) | AGA (aU/mL) | AGG (aU/mL) | DGP-tTG |
| − | 0-2.9 | 0-2.4 | 0-11.9 | 0-11.9 |
| +/− | 3-5.9 | 2.5-3.9 | 12-20 | 12-20 |
| + | 6-20.9 | 4.0-20 | 21-40 | 21-40 |
| ++ | 21-50 | 21-80 | 41-100 | 41-100 |
| +++ | ≧51 | ≧81 | ≧101 | ≧101 |

### Example 2

### Relationship Between Years on Gluten-Free Diet and the Response to Gluten

From the data presented in Table 2 of Example 1, no firm conclusion could be drawn regarding the efficacy of the enzyme therapy as applied in the clinical trial. Neither the intestinal histopathology (Marsh scores), nor the celiac disease specific antibodies anti-endomysium IgA (EMA), tissue transglutaminase IgA (tTGA), anti-gliadin IgA (AGA) and IgG (AGG) show a clear response to the oral intake of the proline-specific protease. However, if the same data are presented according to the present invention, the outcome of the trial allows some firm conclusions.

TABLE 3
Results of the trial described in Example 1 specifying the years that
patients are on a gluten-free diet (GFD) in relation to the various clinical
parameters observed.

| | Marsh | | Marsh |
|---|---|---|---|
| Years on a | response in | Years on a | response in |
| GFD | placebo group | GFD | AN-PEP group |
| 22 | 1 | 6 | −1 |
| 2 | 1 | 4 | 0 |
| 3 | 2 | 11 | 0 |
| 3 | 1 | 10 | 1 |
| 40 | 0 | 8 | 0 |
| 20 | 0 | 12 | 0 |
| 9 | 1 | 7 | 0 |
| | tTGA response | | tTGA response |
| Years on a | (aU/mL) in | Years on a | (aU/mL) in |
| GFD | placebo group | GFD | AN-PEP group |
| 22 | 0 | 6 | 0 |
| 2 | 0.3 | 4 | 0.2 |
| 3 | 1.3 | 11 | 0 |
| 3 | 3.2 | 10 | 0 |
| 40 | 0 | 8 | 0 |
| 20 | 0 | 12 | 0 |
| 9 | 0.4 | 7 | 0 |
| | AGA response | | AGA response |
| Years on a | (aU/mL) in | Years on a | in AN-PEP |
| GFD | placebo group | GFD | group |
| 22 | 0 | 6 | 1 |
| 2 | 43.6 | 4 | 2 |
| 3 | 0.6 | 11 | 1.8 |
| 3 | 0.4 | 10 | 1.8 |
| 40 | 0.3 | 8 | 0.4 |
| 20 | −0.6 | 12 | 0.2 |
| 9 | 0.4 | 7 | −0.4 |
| | AGG response | | AGG response |
| Years on a | (aU/mL) in | Years on a | (aU/mL) in |
| GFD | placebo group | GFD | AN-PEP group |
| 22 | 0 | 6 | 1 |
| 2 | 34.6 | 4 | 1.5 |
| 3 | 2.8 | 11 | 23.3 |
| 3 | 3 | 10 | −0.1 |
| 40 | −1 | 8 | 0.3 |
| 20 | 0 | 12 | −0.2 |
| 9 | 0.4 | 7 | −1 |
| | DGP-tTG | | DGP-tTG |
| | response | | response |
| Years on a | (aU/mL) in | Years on a | (aU/mL) in |
| GFD | placebo group | GFD | AN-PEP group |
| 22 | 1.8 | 6 | 22.7 |
| 2 | 7.6 | 4 | 1.7 |
| 3 | 70.2 | 11 | 12.2 |
| 3 | 13.8 | 10 | 9.7 |
| 40 | 5.3 | 8 | 3.4 |
| 20 | 4 | 12 | 7.1 |
| 9 | 7 | 7 | 2.5 |

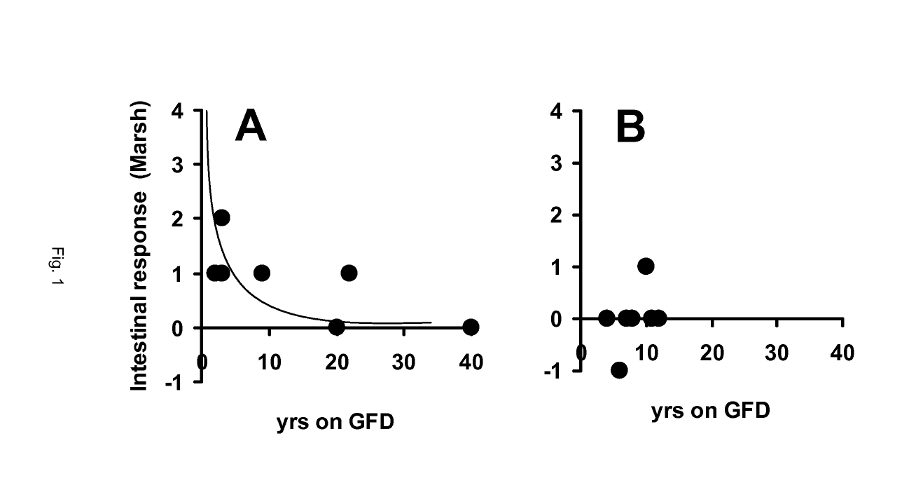

For example, in FIG. 1 we illustrate the relationship between the years spent on a gluten-free diet for each individual patient and his/her response to gluten expressed as change in intestinal histopathology (scored by Marsh) from baseline to the end of the 3^{rd} phase. FIG. 1A shows the Marsh response to gluten on placebo, and FIG. 1B shows the Marsh response to gluten on AN-PEP treatment. FIG. 1A shows that of the 7 subjects who received gluten with placebo, those using a gluten-free diet for only a few years, showed an increase in Marsh and, therefore, responded best to the gluten. FIG. 1B shows that for the 7 subjects receiving gluten plus AN-PEP treatment, no such relationship is present. Therefore, upon comparing the results shown in FIG. 1A with the results shown in FIG. 1B, a preventative effect of AN-PEP consumption can be deducted. Subjects on placebo showing a histopathological response in the intestinal mucosa to gluten (FIG. 1A) were mainly those who had been on a gluten-free diet for 10 years or less, preferably 6 years or less. Since subjects on a gluten-free diet for 10 years or less, preferably 6 years or less respond to gluten, those subjects are of specific interest for testing novel treatment compounds.

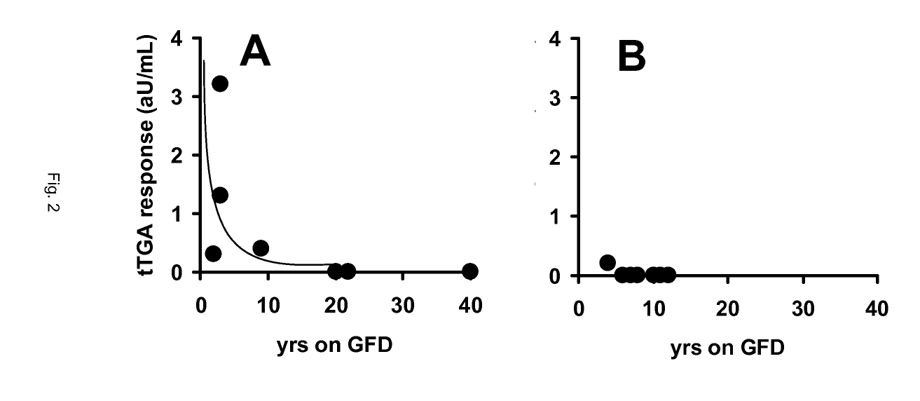

FIG. 2 illustrates the relationship between years on a gluten-free diet and the response to gluten as measured by the change in serum tTGA antibodies as compared to baseline. FIG. 2A shows that only those subjects who had been gluten-free for only a few years showed an increase in serum tTGA whereas those on a gluten-free diet for longer do not or hardly respond. FIG. 2B shows that none of the 7 subjects who received gluten plus AN-PEP treatment respond to gluten. From this it can be deducted that AN-PEP treatment probably prevents the tTGA response to gluten. Subjects on placebo showing a tTGA response to gluten were mainly those who had been on a gluten-free diet for only a few years. Therefore, those subjects are most suitable for inclusion in studies to test efficacy/effectiveness of novel treatment compounds.

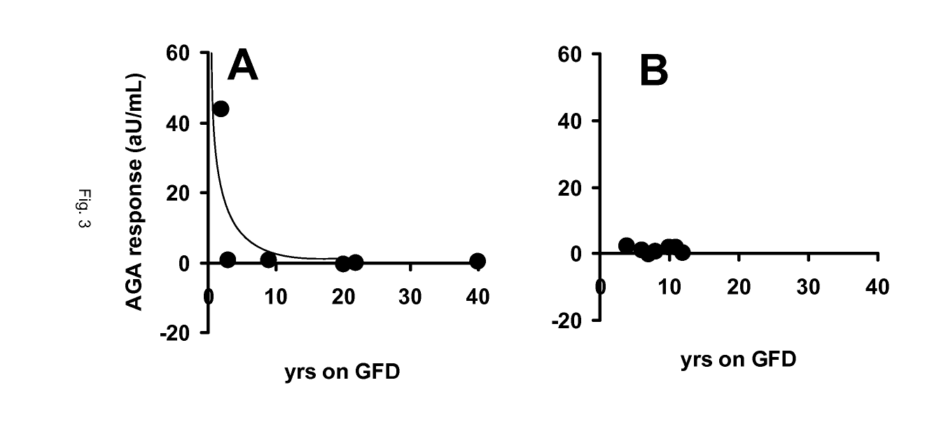

FIG. 3 illustrates the relationship between the change in serum AGA antibodies and years on gluten-free diet from baseline to end of the 3^{rd} phase. FIG. 3A shows that subjects on placebo that respond to gluten as measured by increased serum AGA were mainly those who had been on a gluten-free diet for only a few years. FIG. 3B shows that the consumption of AN-PEP probably prevents a rise in AGA on gluten in such patients.

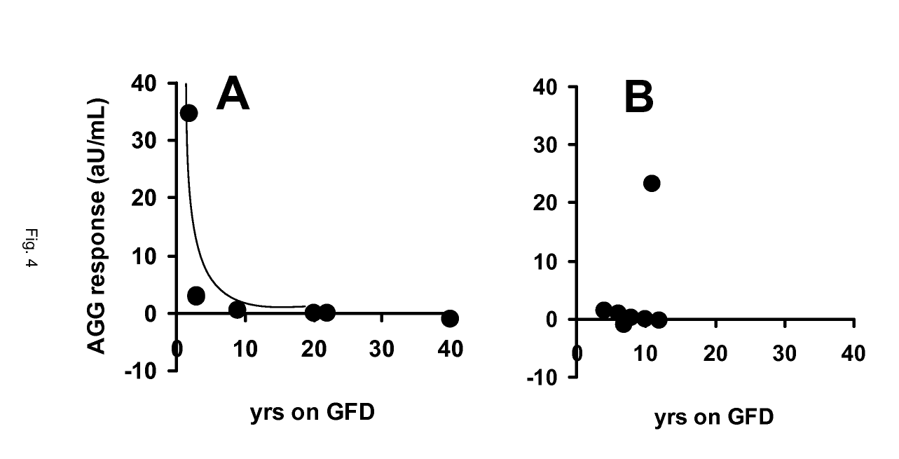

FIG. 4 illustrates the relationship between the change in serum anti-gliadin IgG (AGG) antibodies and years on gluten-free diet from baseline to end of the randomization phase. FIG. 4A shows that subjects on placebo show an AGG response to gluten (FIG. 4A) were mainly those who had been on a gluten-free diet for only a few years. FIG. 4B shows a poor relationship between the 7 subjects who received gluten plus AN-PEP treatment, implying that AN-PEP is probably preventing a rise in AGG on gluten. Since particularly subjects on a gluten-free diet for only a few years respond to gluten, those subjects are of specific interest to test novel treatment compounds.

The data obtained in the present experiment illustrate that responses to a 2-week 7 g daily gluten challenge are small and that only few subjects respond by clinical signs. However, quite surprisingly, particularly those subjects who are on a gluten diet for 10 years or less, preferably 6 years or less show a response on intestinal histopathology by Marsh classification and celiac disease-specific serum antibodies (tTGA, AGA, AGG, and DGP-tTG) and are suitable for testing effectiveness/efficacy of treatment compounds such as AN-PEP.

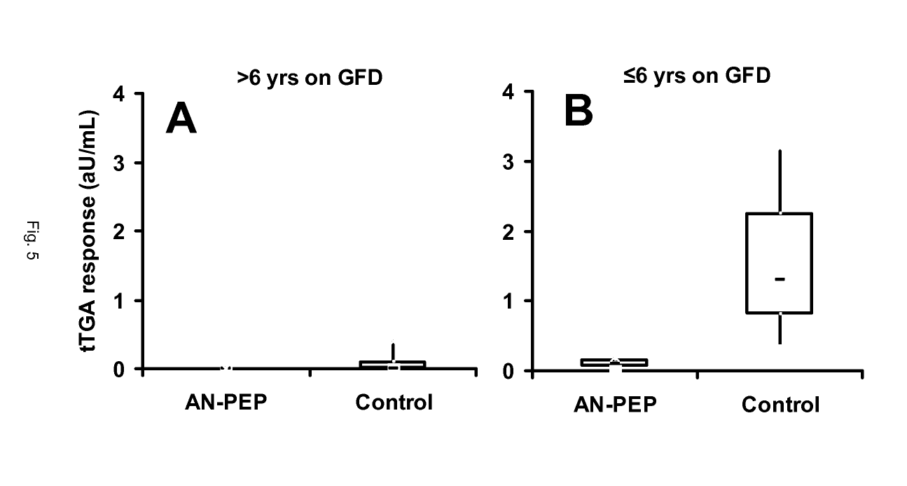

FIG. 5 shows that no difference in tTGA response to gluten could be observed between placebo and AN-PEP treatment if results from patients over 6 years on gluten-free diet are taken into account. However, upon selecting those patients that are on a gluten-free diet for 6 years or less, a significant difference in tTGA response to gluten was observed between the placebo and AN-PEP treatments. This illustrates that selecting patients being 6 years or less on a gluten-free diet is advantageous for properly determining the efficacy of treatments or therapies for celiac disease.

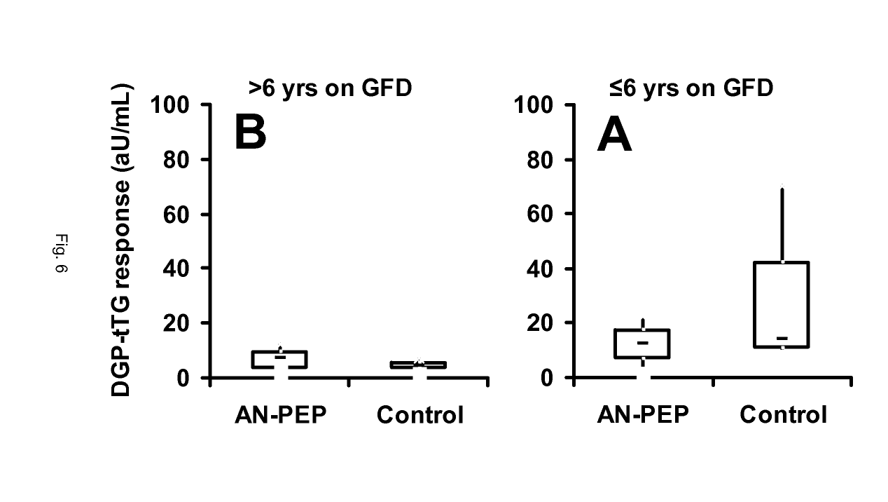

FIG. 6 shows that no difference in DGP-tTG response to gluten could be observed between placebo and AN-PEP treatment when selecting patients on a gluten-free diet for more than 6 years. However, when patients on a gluten-free diet for 6 years or less are selected, a significant treatment effect by AN-PEP on DGP-tTG was observed compared to placebo. Since subjects on a gluten-free diet for 6 years or less respond to gluten, those subjects are of specific interest for testing novel treatment compounds.

### Example 3

### Statistical Evaluation of the Relationship Between Years on Gluten-Free Diet and the Response to Gluten

Because the clinical parameters are not normally distributed, non-parametric tests are more efficient (Hollander and Wolfe, Nonparametric Statistical Methods. New York: John Wiley & Sons, 1973). Therefore, the values were ranked to calculate statistical correlations and differences between treatment groups.

All serological (blood tTGA, AGG, AGA, and DGP-tTG antibodies) and histopathological (intestinal Marsh score) outcome parameters and of the subjects finalizing the randomization phase were ranked in order of magnitude. Observations with equal rank were assigned the average of the ranks for the range they cover. Similarly, subjects were assigned a rank number in order of years on gluten-free diet. Correlations between ranked clinical parameters and ranked years on gluten-free diet were explored using the Spearman Rank Correlation Coefficient (r) to explore the strength of correlations at the different time points. See FIG. 7 for the design of the clinical trial. Time points included baseline (t=0), 1 week (WA), and 2 weeks (t=1) of the safety phase of gluten intake plus AN-PEP treatment as well as at 1 week (t=1½) and 2 weeks (t=2) of the randomization randomization phase of gluten intake plus AN-PEP or placebo treatment. p<0.05 was considered significant.

All statistical analysis was performed using SAS software (SAS Institute, Cary, NC, USA).

Table 4 shows the rank correlation coefficients for (almost) significant relationships between years on gluten-free diet and different clinical parameters in the total and control group at different time points.

A significant negative rank correlation was observed between the subjects' years on gluten-free diet and the Marsh scores at the end of the randomization phase for all subjects the total group (Table 4). This correlation was even stronger in the group receiving gluten with placebo treatment (Table 4). Significant negative rank correlations were observed between the subjects' years on gluten-free diet and serum tTGA antibody levels at baseline, during and at the end of the randomization study period (Table 4). These correlations between time on gluten-free diet and response to gluten were stronger in the placebo group (Table 4). In addition, a significant negative rank correlation was observed in the whole group between subjects' years on gluten-free diet and serum AGG antibody levels at the end of the randomization phase (Table 4). This correlation was even stronger in the placebo group. For serum AGA antibody levels, no significant rank correlations were observed. The years a subject was adhering to a gluten-free diet was significantly inversely rank correlated to the response to gluten as measured by the sensitive DGP-tTG antibody test after 2-weeks of gluten challenge period in the randomization phase in the placebo group. This indicates that the subjects' gluten responsiveness decreases with years on a gluten-free diet.

TABLE 4
Rank correlation coefficients for relationships between years on
gluten-free diet and different clinical parameters in the total and control
group at the end of the randomization phase.

| | Total group | | | |
|---|---|---|---|---|
| | (Gluten + AN-PEP | | Placebo group | |
| | or placebo) | | (Gluten + placebo) | |
| | Rank correlation | | Rank correlation | |
| Parameter | coefficient (r) | p-value | coefficient (r) | p-value |
| Marsh | −0.58 | 0.03* | −0.810 | 0.0271* |
| tTGA | −0.67 | 0.009* | −0.84 | 0.02* |
| AGA | −0.51 | 0.06 | −0.69 | 0.09 |
| AGG | −0.81 | 0.0004* | −0.98 | <0.0001* |
| DGP-tTGA | −0.39 | 0.1740 | −0.774 | 0.04* |

p < 0.05 was considered significantly different.
*Significantly different correlation

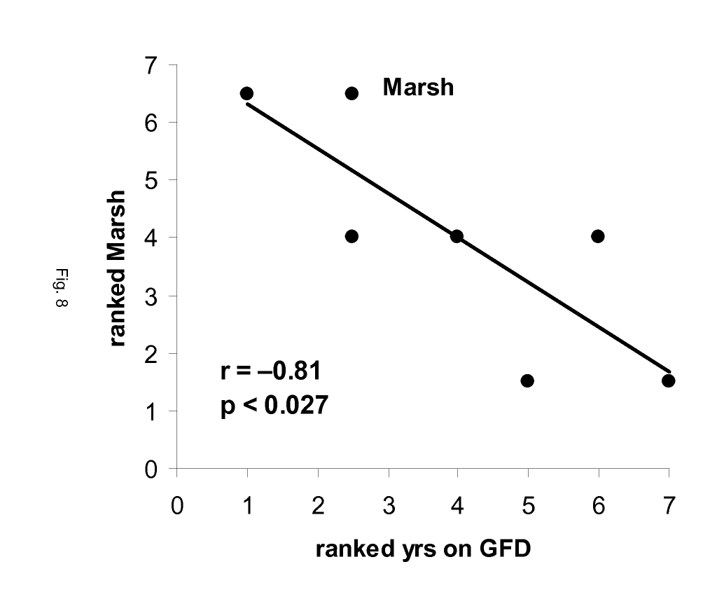

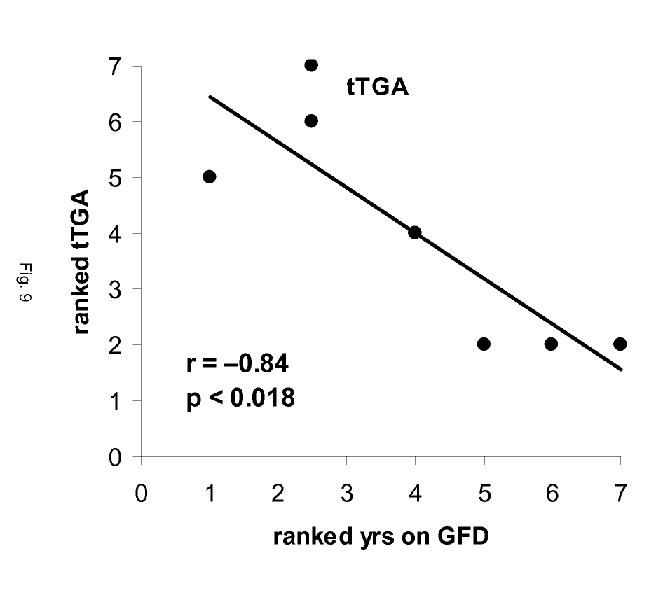

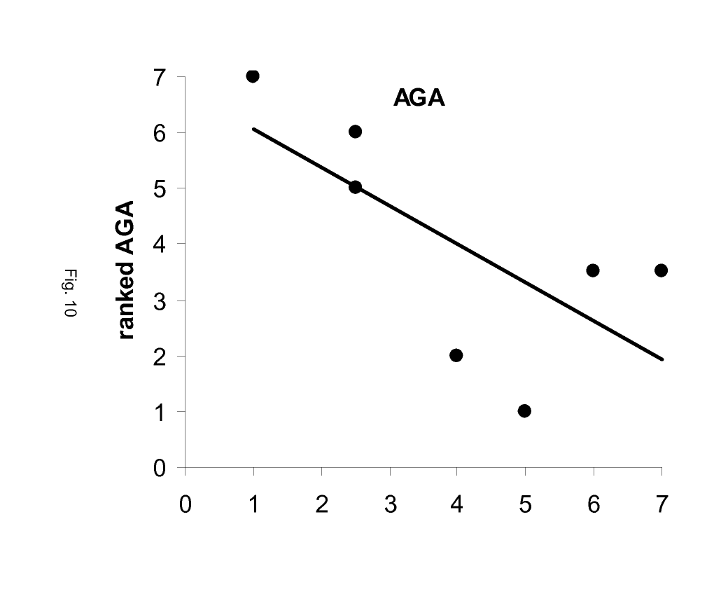

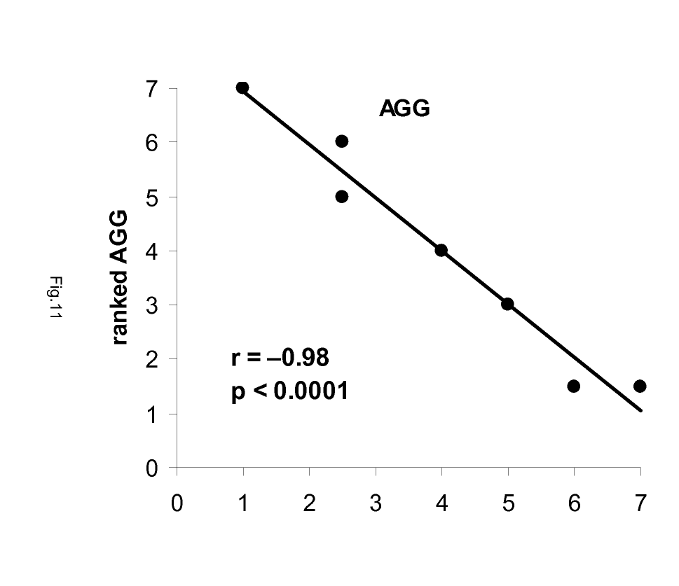

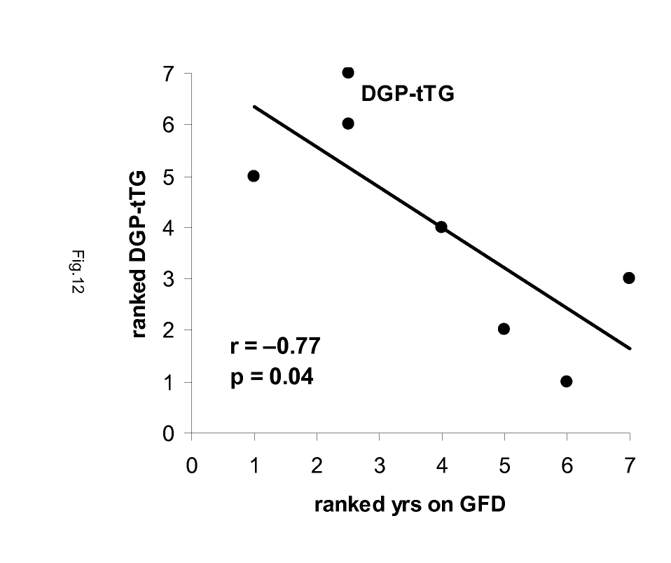

FIGS. 8 to 12 show the relationships in the placebo group at the end of the randomization phase after gluten plus placebo intake, between subjects' years on gluten-free diet and different clinical parameters (Marsh scores, serum tTGA, AGA, AGG, and DGP-tTG antibodies).

The observed statistically significant negative relationships between the subjects' years on a gluten-free diet and the subjects' clinical response to gluten demonstrates that celiac patients that are on a gluten-free diet for relatively short time respond better to gluten, and, hence, are a more suitable population to test novel treatment compound.

We tested the hypothesis that celiac patients that are on a gluten-free diet for relatively short time are a better population to recruit in order to demonstrate effects of a treatment compared to a reference compound, as they respond better to gluten. For that purpose, we selected subjects participating in our clinical trial that were on a gluten-free diet for less than 10 years (n=4 in the AN-PEP and n=4 in the placebo group). Within this subpopulation, we compared the clinical response to 7 g daily gluten intake between AN-PEP and placebo treatment during and at the end of the randomization phase. Since clinical parameters were not normally distributed, absolute values were ranked.

Table 5 shows the absolute values and ranks of the different clinical parameters in subjects previously on a gluten-free diet for less than 10 years for which significant correlations with time on gluten-free diet were found.

TABLE 5
Individual absolute values and ranks of clinical parameters in subjects that were
on a gluten-free diet for less than 10 years.

| | | Marsh | | tTGA | | tTGA | | AGA | | AGG | | DGP-tTG | |
|---|---|---|---|---|---|---|---|---|---|---|---|---|---|
| | | score | | (aU/mL) | | (aU/mL) | | (aU/mL) | | (aU/mL) | | (aU/mL) | |
| | | End of 3^{rd} | | Halfway | | End of 3^{rd} | | End of 3^{rd} | | End of 3^{rd} | | End of 3^{rd} | |
| | GFD | phase | | 3^{rd} phase | | phase | | phase | | phase | | phase | |
| treatment | (yr) | Abs | Rank | Abs | Rank | Abs | Abs | Abs | Rank | Abs | Rank | Abs | Rank |
| AN-PEP | 6 | 1 | 4.0 | 0 | 2.0 | 0 | 2.0 | 1.5 | 3.5 | 1.5 | 2.0 | 22.7 | 7.0 |
| AN-PEP | 4 | 1 | 4.0 | 1.5 | 7.0 | 1.2 | 6.0 | 2.5 | 7.0 | 2.6 | 4.0 | 1.7 | 1.0 |
| AN-PEP | 8 | 1 | 4.0 | 0 | 2.0 | 0 | 2.0 | 1.5 | 3.5 | 1.7 | 3.0 | 3.4 | 3.0 |
| AN-PEP | 7 | 0 | 1.0 | 0 | 2.0 | 0 | 2.0 | 0.9 | 1.0 | 2.7 | 5.0 | 2.5 | 2.0 |
| Placebo | 2 | 3 | 7.5 | 0.5 | 4.5 | 0.8 | 5.0 | 46.3 | 8.0 | 36.2 | 8.0 | 7.6 | 5.0 |
| Placebo | 3 | 3 | 7.5 | 1 | 6.0 | 1.9 | 7.0 | 1.8 | 5.0 | 6.9 | 7.0 | 70.2 | 8.0 |
| Placebo | 3 | 1 | 4.0 | 4.7 | 8.0 | 7.3 | 8.0 | 1.9 | 6.0 | 5.1 | 6.0 | 13.8 | 6.0 |
| Placebo | 9 | 1 | 4.0 | 0.5 | 4.5 | 0.4 | 4.0 | 1.2 | 2.0 | 1.1 | 1.0 | 7.4 | 4.0 |

The sum of ranks was compared between the AN-PEP and placebo groups using the non-parametric one-sided Wilcoxon test (Hollander and Wolfe, Nonparametric Statistical Methods. New York: John Wiley & Sons, 1973). A difference was considered significant when p<0.05. Table 6 shows the median values and the sum of ranks and of different clinical parameters for the two groups and the probability that the sum of ranks is different between the groups. The sum of ranks was lower in the AN-PEP than in the placebo group for all parameters (Table 6). At the end of the 3^{rd} phase after 2 weeks of gluten challenge, the sum of ranks of the serum tTGA antibodies was significantly lower in the AN-PEP group than in the placebo group. At the end of the 3^{rd} phase, the sum of ranks of the intestinal histopathological Marsh scores was also significantly lower in the AN-PEP group than in the placebo group.

This statistical data analysis substantiates that when selecting celiac patients that are on a gluten-free diet for less than 10 years, a treatment effect of AN-PEP can be demonstrated for some clinical parameters.

TABLE 6
Medians and sum of ranks of clinical parameters in subjects
that were on a gluten-free diet for less than 10 years. Sum
of ranks were compared between groups using Wilcoxon
test and p < 0.05 considered significantly different.

| | | Median | | Sum of ranks | | |
|---|---|---|---|---|---|---|
| | | AN- | | AN- | | |
| | | PEP | Placebo | PEP | Placebo | |
| Parameter | Visit | (n = 4) | (n = 4) | (n = 4) | (n = 4) | p-value |
| Marsh | End of 3^{rd} | 1.0 | 2.0 | 13 | 23 | 0.05* |
| | phase | | | | | |
| tTGA | Halfway 3^{rd} | 0.0 | 0.85 | 13 | 23 | 0.07 |
| | phase | | | | | |
| tTGA | End of 3^{rd} | 0.0 | 1.45 | 12 | 24 | 0.04* |
| | phase | | | | | |
| AGA | End of 3^{rd} | 1.5 | 1.9 | 15 | 21 | 0.19 |
| | phase | | | | | |
| AGG | End of 3^{rd} | 2.2 | 6.0 | 14 | 22 | 0.12 |
| | phase | | | | | |
| DGP-tTG | End of 3^{rd} | 2.5 | 5.5 | 13 | 23 | 0.07 |
| | phase | | | | | |

*Significantly different from placebo

1. A method to determine effectiveness of a compound or composition in treatment of celiac disease or gluten intolerance, the method comprising:
(a) administering the compound or composition to one or more human patients who were on a gluten-free diet for 10 years or less,
(b) challenging with gluten the one or more patients who were administered the compound or composition, and
(c) determining whether there is a change in condition of celiac disease or gluten intolerance of the one or more patients that results from administering the compound or composition and then challenging with gluten;
whereby no significant change in condition indicates that the compound or composition would be effective in treating celiac disease or gluten intolerance.

2. A method to determine effectiveness of a compound or composition in treatment of celiac disease or gluten intolerance, the method comprising:
(a) selecting one or more human patients having celiac disease or gluten intolerance, wherein said patients
(i) have a stable condition with respect to celiac disease or gluten intolerance and
(ii) were on a gluten-free diet for 10 years or less;
(b) determining condition of illness of the one or more selected patients before administration of the compound or composition, and then challenge with gluten;
(c) administering the compound or composition to the one or more selected patients;
(d) challenging with gluten the one or more selected patients who were administered the compound or composition;
(e) determining condition of illness of the one or more selected patients after administration of the compound or composition, and then challenge with gluten; and
(f) comparing the condition of illness of the one or more selected patients before and after administration of the compound or composition, and then challenge with gluten;
wherein a substantially unchanged condition of illness of the one or more selected patients after administration of the compound or composition and then challenge with gluten as compared to the condition of illness before administration of the compound or composition and then challenge with gluten indicates the compound or composition would be effective in treating celiac disease or gluten intolerance.

3. A method to improve analysis of data from a clinical trial; whereby one or more human patients are (a) administered a compound or composition, (b) challenged with gluten, and (c) diagnosed for a change in condition of celiac disease or gluten intolerance that results from administering the compound or composition and then challenging with gluten; wherein said method further comprises at least:
including in the clinical trial only the one or more patients who were on a gluten-free diet for 10 years or less, or
excluding from the clinical trial all of the one or more patients who were on a gluten-free diet for more than 10 years, or
including only data in the analysis obtained by diagnosing the one or more patients who were on a gluten-free diet for 10 years or less, or
excluding all data from the analysis obtained by diagnosing the one or more patients who were on a gluten-free diet for more than 10 years.

4. The method according to claim 1, wherein the one or more patients were on a gluten-free diet for more than 3 months.

5. The method according to claim 1, wherein the one or more patients were on a gluten-free diet for 8 years or less.

6. The method according to claim 1, wherein the change in condition of illness of the one or more patients between before and after administration of the compound or composition is compared with a change in condition of illness of the one or more patients between before and after administration of a control compound or composition, wherein the control compound or composition optionally comprises a placebo.

7. The method according to claim 1, wherein said compound or composition comprises an enzyme, optionally an enzyme which acts on gluten, and optionally a prolyl-specific endoprotease.

8. The method according to claim 1, wherein said compound or composition comprises a compound or composition which can improve intestinal barrier function, optionally a zonulin receptor inhibitor or compound or composition having chemokine receptor antagonist action.

9. The method according to claim 1, wherein the determining or diagnosis of condition of illness uses one or more criteria selected from the group consisting of Marsh classification, antibodies such as anti-tTGA antibody, measurement of intestinal permeability such as by sugar test, presence and activity of T cells in blood and intestinal tissue, and symptoms analysis.

10. The method according to claim 1, which is a clinical trial.

11. The method according to claim 1, which is a treatment trial, quality of life trial, or compassionate use trial.

12. The method according to claim 1, wherein the change in condition of celiac disease or gluten intolerance is determined in at least 5 human patients.

13. A compound or composition capable of being used in a method to determine effectiveness of a compound or composition in treatment of celiac disease or gluten intolerance comprising:
(a) administering the compound or composition and a gluten challenge to one or more human patients who were on a gluten-free diet for 10 years or less;
(b) challenging with gluten the one or more patients who were administered the compound or composition; and
(c) determining whether there is a change in condition of celiac disease or gluten intolerance of the one or more patients that results from administering the compound or composition and then challenging with gluten;
whereby no significant change in condition indicates that the compound or composition would be effective in treating celiac disease or gluten intolerance.

14. A compound or composition capable of being used in a method to determine the effectiveness of a compound or composition in the treatment of celiac disease or gluten intolerance comprising:
(a) selecting one or more human patients having celiac disease or gluten intolerance, wherein said patients
(i) have a stable condition with respect to celiac disease or gluten intolerance and
(ii) were on a gluten-free diet for 10 years or less;
(b) determining condition of illness of the one or more selected patients before administration of the compound or composition, and then challenge with gluten;
(c) administering the compound or composition to the one or more selected patients;
(d) challenging with gluten the one or more selected patients who were administered the compound or composition;
(e) determining condition of illness of the one or more selected patients after administration of the compound or composition, and then challenge with gluten; and
(f) comparing the condition of illness of the one or more selected patients before and after administration of the compound or composition, and then challenge with challenge;
wherein a substantially unchanged condition of illness of the one or more selected patients after administration of the compound or composition and then challenge with gluten as compared to the condition of illness before administration of the compound or composition and then challenge with gluten indicates the compound or composition would be effective in treating celiac disease or gluten intolerance.

15. A compound or composition capable of being used according to claim 13, wherein the one or more patients were on a gluten-free diet for more than 3 months.

16. A compound or composition capable of being used according to claim 13, wherein the one or more patients were on a gluten-free diet for 8 years or less.

17. A compound or composition capable of being used according to claim 13, wherein the change in condition of illness of the one or more patients between before and after administration of the compound or composition is compared with the change in condition of illness of the one or more patients between before and after administration of a control compound or composition, wherein the control compound or composition optionally comprises a placebo.

18. A compound or composition capable of being used according to claim 13 wherein the compound or composition comprises an enzyme, optionally an enzyme which acts on gluten, and optionally a prolyl-specific endoprotease.

19. A compound or composition capable of being used according to claim 13, wherein the compound or composition comprises a compound or composition which can improve intestinal barrier function, optionally a zonulin receptor inhibitor or compound or composition having chemokine receptor antagonist action.

20. A compound or composition capable of being used according to claim 13, wherein the determining condition of illness uses one or more criteria selected from the group consisting of Marsh classification, antibodies which can optionally comprise anti-tTGA antibody, measurement of intestinal permeability optionally by sugar test, presence and activity of T cells in blood and intestinal tissue, and symptoms analysis.

21. A compound or composition capable of being used according to claim 13, in a clinical trial.

22. A compound or composition capable of being used according to claim 13, in a treatment trial, quality of life trial, or compassionate use trial.

23. A compound or composition capable of being used according to claim 13, wherein the change in condition of celiac disease or gluten intolerance is determined in at least 5 human patients.

24. An interpretation of a clinical trial for testing the effectiveness of a compound or composition in treatment of celiac disease or gluten intolerance, by using the results from one or more human patients who were on a gluten-free diet for 10 years or less; or optionally by using only results from human patients who were on a gluten-free diet for 10 years or less.

25. A health claim, disease reduction claim, or label for a compound or composition for treatment of celiac disease or gluten intolerance or labeling a compound or composition for treatment of celiac disease or gluten intolerance which is based on a clinical trial for determining effectiveness of a compound or composition in treatment of celiac disease or gluten intolerance, by using results from one or more human patients who were on a gluten-free diet for 10 years or less; or optionally by using only results from one or more human patients who were on a gluten-free diet for 10 years or less.

26. The health claim, disease reduction claim, label, or labeling according to claim 25, capable of being used to indicate, advertise, or promote a compound or composition for treatment of celiac disease or gluten intolerance.

27. An exclusion or inclusion criteria of a clinical trial capable of being used to determine the effectiveness of a compound or composition for treatment of celiac disease or gluten intolerance comprising one or more human patients who were on a gluten-free diet for 10 years or less; or optionally only human patients who were on a gluten-free diet for 10 years or less.
